(19) United States
(12) Patent Application Publication
(10) Pub. No.: US 2015/0017242 A1
(43) Pub. Date: Jan. 15, 2015

(54) Bilayer Tablet of Dronedarone

(71) Applicant: Lupin Limited, Mumbai (IN)

(72) Inventors: Ashish Ashokrao Deshmukh, Pune (IN); Hemant Bhalerao, Pune (IN); Pravin Meghrajji Bhutada, Pune (IN); Sajeev Chandran, Pune (IN); Shirishkumar Kulkarni, Pune (IN)

(21) Appl. No.: 14/379,608

(22) PCT Filed: Feb. 14, 2013
(86) PCT No.: PCT/IB2013/051182

§ 371 Date: Aug. 19, 2014

(30) Foreign Application Priority Data
Feb. 20, 2012 (IN) 160/KOL/2012

(51) Int. Cl. A61K 31/343; A61K 9/24

(52) U.S. Cl. CPC A61K 31/343; A61K 9/209
USPC 424/472; 514/469; 424/474

(57) ABSTRACT

The present invention relates to multi component composition of dronedarone or pharmaceutically acceptable salts, esters, metabolites, prodrugs or enantiomers thereof and controlled release polymers. The use of multi component composition of Dronedarone would improve the bioavailability and the patient compliance with reduction in number of dosages to be taken per day.

### FIELD OF THE INVENTION

The present invention describes a multi component composition of dronedarone which maintains a therapeutically effective blood concentration with once a day administration.

### BACKGROUND OF THE INVENTION

Cardiac arrhythmia is a term for any large and heterogeneous group of conditions in which there is abnormal electric activity in the heart. An arrhythmia is a disorder of the heart rate (pulse) or heart rhythm, such as beating too fast (tachycardia), too slow (bradycardia), or irregularly. Arrhythmias can be life-threatening medical emergencies which can result in cardiac arrest and sudden death.

Normally, the four chambers of the heart contract in very specific and coordinated manner. The electrical impulse that signals the heart to contract in a synchronized manner begins in the sinoatrial node (SA node) which is heart's natural pacemaker. The signal leaves the SA node and travels through the two upper chambers (atria). Then the signal passes through the atrioventricular node (AV node). Finally it passes through the lower chambers (ventricles). This path enables the chambers to contract in a coordinated fashion. Problems can occur anywhere along this conduction system, causing various arrhythmias. The examples include: Bradycardia—a slow heart rate due to problems with the SA node's pacemaker ability, or an interruption in energy movement (conduction) through the natural electrical pathways of the heart. Supraventricular tachycardia—a fast heart rate that originates in the upper chambers (atria). The most common are atrial fibrillation or flutter (a rapid heart rate that is not regular). and atrioventricular nodal reentry tachycardia (AVNRT). Ventricular tachycardia—a fast heart rate that originates in the lower chambers (ventricles).

The method of cardiac rhythm management depends firstly on whether or not the affected person is stable or unstable. Treatments may include physical maneuvers, medications, electricity conversion, or electro or cryo cautery. When an arrhythmia is serious, urgent treatment may be required to restore a normal rhythm. This may include: electrical “shock” therapy (defibrillation or cardioversion), implanting a temporary pacemaker to interrupt the arrhythmia medications given through a vein (intravenous). Medications are generally used to prevent and/or manage arrhythmia. There are many classes of antiarrhythmic medications, with different mechanisms of action which include Sodium Channel Blockers (Class I) e.g. Class IA—Quinidine (Quinidex), Procainamide (Pronestyl), Disopyramide (Norpace); Class IB—Lidocaine (Xylocaine), Tocainide (Tonocard), Mexiletine (Mexitil); Class IC—Encainide (Enkaid), Flecainide (Tambocor); Beta-Adrenergic Blockers (Class II)—Propranolol (Inderal), Acebutolol (Sectral), Esmolol (Brevibloc), Sotalol (Betapace); Drugs that Prolong Repolarization (Class III)—Dronedarone (Multaq), Amiodarone (Cordarone); Calcium Channel Blockers (Class IV)—Verapamil (Calan, Isoptin), Diltiazem (Cardizem), Mebefradil (Posicor); Miscellaneous-Adenosine (Adenocard), Digoxin (Lanoxin).

Dronedarone hydrochloride is N-{2-butyl-3-[4-(3-dibutylaminopropoxy)benzoyl]benzofuran-5-yl}methane sulfonamide, hydrochloride.

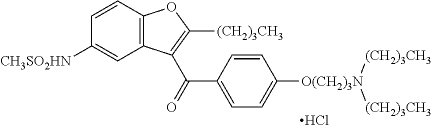

The effects of Dronedarone most likely result from its electrophysiological properties belonging to all four Vaughan-Williams classes. Dronedarone is a multichannel blocker inhibiting the potassium currents (including IK (Ach), IKur, IKr, IKs) and thus prolonging cardiac action potential and refractory periods (Class III). It also inhibits the sodium currents (Class Ib) and the calcium currents (Class IV). It non-competitively antagonizes adrenergic activities (Class II). Dronedarone works by altering currents passing through potassium, sodium, and calcium channels, thereby prolonging conduction in the heart. This helps maintain a regular heart rhythm or sinus rhythm and slows the heart rate.

The available dosage for Dronedarone is 400 mg oral tablet to be administered twice a day with meals. Dronedarone hydrochloride (Multaq®; Sanofi-Aventis) was approved to reduce the risk of cardiovascular hospitalization in patients with paroxysmal or persistent atrial fibrillation (AF) or atrial flutter (AFL), with a recent episode of AF/AFL and associated cardiovascular risk factors (i.e., age >70, hypertension, diabetes, prior cerebrovascular accident, left atrial diameter ≧50 mm or left ventricular ejection fraction [LVEF]<40%), who are in sinus rhythm or who will be cardioverted). Dronedarone is also found to be useful in prevention of stroke or transient ischemic attack, prevention of permanent atrial fibrillation, prevention of cardioversion, regulating potassium levels in blood, for prevention of cardiac arrhythmia and increased creatinine level, reducing death rate after infarction and reducing death rate after infarction. The primary advantage of dronedarone is its comparatively lower side-effect profile vis-à-vis amiodarone. Due to high presystemic first pass metabolism the absolute oral bioavailability is only 4% (fasting) which increases to approx. 15% when administered with a high fat meal.

U.S. Pat. No. 5,223,510 assigned to Sanofi discloses dronedarone specifically.

U.S. Pat. No. 7,323,493 assigned to Sanofi Aventis relates to a solid pharmaceutical composition for oral administration characterized in that it comprises a benzofuran derivative with antiarrhythmic activity, or one of the pharmaceutically acceptable salts thereof, as an active principle, and a pharmaceutically acceptable nonionic hydrophilic surfactant optionally in combination with one or more pharmaceutical excipients.

US 2007/0243257 filed by Sanofi Aventis relates to a solid pharmaceutical composition comprising a solid dispersion containing at least one active principle and a pharmaceutically acceptable polymer matrix, characterized in that said pharmaceutically acceptable polymer matrix comprises a blend of (i) polydextrose, in the form of a continuous polydextrose phase, in order to promote the disintegration of the composition in an aqueous medium, and (ii) at least one polymer other than polydextrose, in the form of a continuous phase of this polymer, whereby the polydextrose is in a concentration of at least 20 wt % and the at least one polymer other than polydextrose is in a concentration of at least 20 wt % in relation to the total weight of said pharmaceutically acceptable polymer matrix.

US 2008/0139645 filed by Sanofi Aventis relates to a solid pharmaceutical composition for oral administration characterized in that it comprises a benzofuran derivative with antiarrhythmic activity, or one of the pharmaceutically acceptable salts thereof, as an active principle, and a pharmaceutically acceptable nonionic hydrophilic surfactant optionally in combination with one or more pharmaceutical excipients.

WO 2011/135581 filed by Cadila Healthcare relates to a pharmaceutical composition of dronedarone or salts thereof, characterized in that said composition does not contain surfactant(s), preferably, nonionic hydrophilic surfactant(s) the invention also relates to process or making such compositions.

WO 2011/135582 filed by Cadila Healthcare relates to a pharmaceutical composition comprising dronedarone or pharmaceutically acceptable salts thereof and one or more surfactant/s other than nonionic hydrophilic surfactants.

Although number of approaches have been disclosed in the prior art for preparing a composition comprising dronedarone none describe a multi component composition of dronedarone to provide once a day administration. There exists a need for multi component composition of Dronedarone which controls the release of Dronedarone in such a manner that therapeutically effective concentration is maintained in the blood for an extended period of time keeping the drug concentration in the blood substantially constant. Dronedarone has low solubility in aqueous media and/or at low pH, also at higher pH condition it precipitates out. As a result it has low in-vivo bioavailability, the use of multi component composition of Dronedarone would improve the bioavailability and the patient compliance with reduction in number of dosages to be taken per day.

It has been observed, surprisingly that it is possible to modify the release profile of Dronedarone hydrochloride, using multi component composition comprising prior release component and controlled release component comprising dronedarone for obtaining a controlled release of Dronedarone up to extended period of time, preferably up to 20 hours or more.

The multi component pharmaceutical compositions of dronedarone are administered less frequently and may alleviate the above disclosed problems associated with conventional immediate release compositions.

### SUMMARY OF THE INVENTION

In accordance, one embodiment discloses a multi component composition of dronedarone comprising a prior release component and a controlled release component wherein prior release component comprises about 20% to about 60% by weight of dronedarone based on total amount of dronedarone in the multi component composition.

Yet another embodiment discloses a multi component composition of dronedarone comprising: a) at least one controlled release component comprising dronedarone and control release polymer; b) at least one component comprising control release polymer, characterized in that the component is free of dronedarone; an optional prior release component coated on components a) and b) comprising dronedarone.

Yet another embodiment discloses a multi component composition of dronedarone comprising: a) prior release component comprising dronedarone and pharmaceutically acceptable excipient wherein prior release component comprises about 20% to about 60% by weight of dronedarone based on total amount of dronedarone in the multi component composition; b) controlled release component comprising control release polymer; c) an optional component comprising controlled release polymer between the components a) and b)

Yet another embodiment discloses a multi component composition of dronedarone comprising a prior release component and a controlled release component wherein prior release component comprises about 20% to about 60% by weight of dronedarone based on total amount of dronedarone in the multi component composition and demonstrates a plasma exposure of a dronedarone over a given time period which is equivalent to two immediate release tablet of dronedarone

Yet another embodiment discloses a multi component composition of dronedarone comprising: a) at least one controlled release component comprising dronedarone and control release polymer; b) at least one component comprising control release polymer, characterized in that the component is free of dronedarone; c) an optional prior release component coated on components a) and b) comprising dronedarone and demonstrates a plasma exposure of dronedarone over a given time period which is equivalent to two immediate release tablet of dronedarone

Yet another embodiment discloses a multi component composition of dronedarone comprising: a) prior release component comprising dronedarone and pharmaceutically acceptable excipient wherein prior release component comprises about 20% to about 60% by weight of dronedarone based on total amount of dronedarone in the multi component composition; b) controlled release component comprising control release polymer; c) an optional component comprising controlled release polymer between the components a) and b) and demonstrates a plasma exposure of dronedarone over a given time period which is equivalent to two immediate release tablet of dronedarone.

### BRIEF DESCRIPTION OF THE DRAWINGS

FIG. 1 shows the release profiles of dronedarone for examples 1, 2, and 3 according to measurements under the USP basket method of 100 rpm in 1000 ml phosphate buffer at pH 4.5 at 37° C.

FIG. 2 shows the comparative dronedarone plasma concentration (ng/ml) over a period of 24 hours for (a) single dose of tablet prepared according to example 1 and (b) Multaq® 400 mg immediate release tablet administered twice daily under fed condition.

FIG. 3 shows the comparative dronedarone plasma concentration (ng/ml) over a period of 24 hours for (a) single dose of tablet prepared according to example 2 and (b) Multaq® 400 mg immediate release tablet administered twice daily under fed condition.

FIG. 4 shows the comparative dronedarone plasma concentration (ng/ml) over a period of 24 hours for (a) single dose of tablet prepared according to example 3 and (b) Multaq® 400 mg immediate release tablet administered twice daily under fed condition.

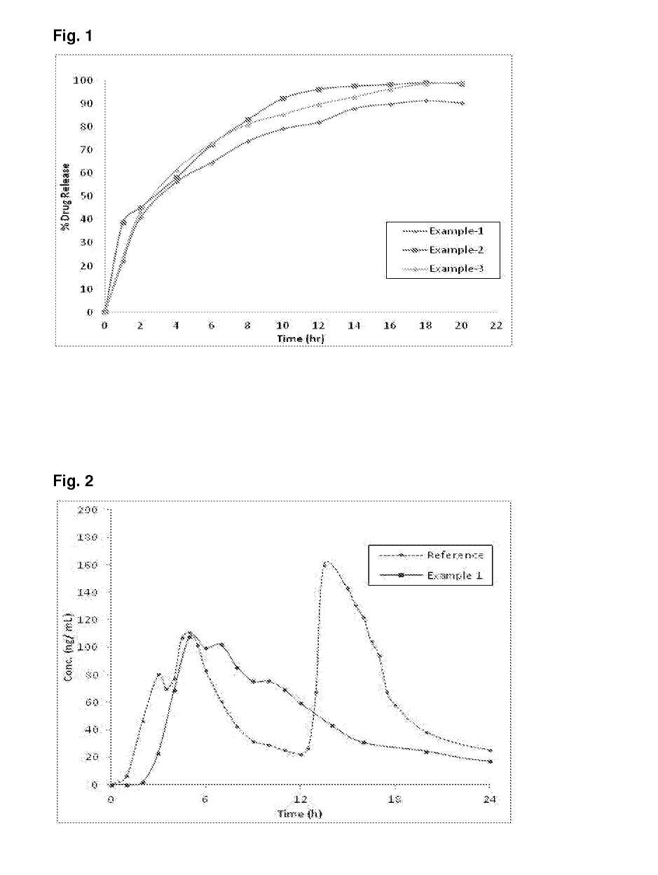

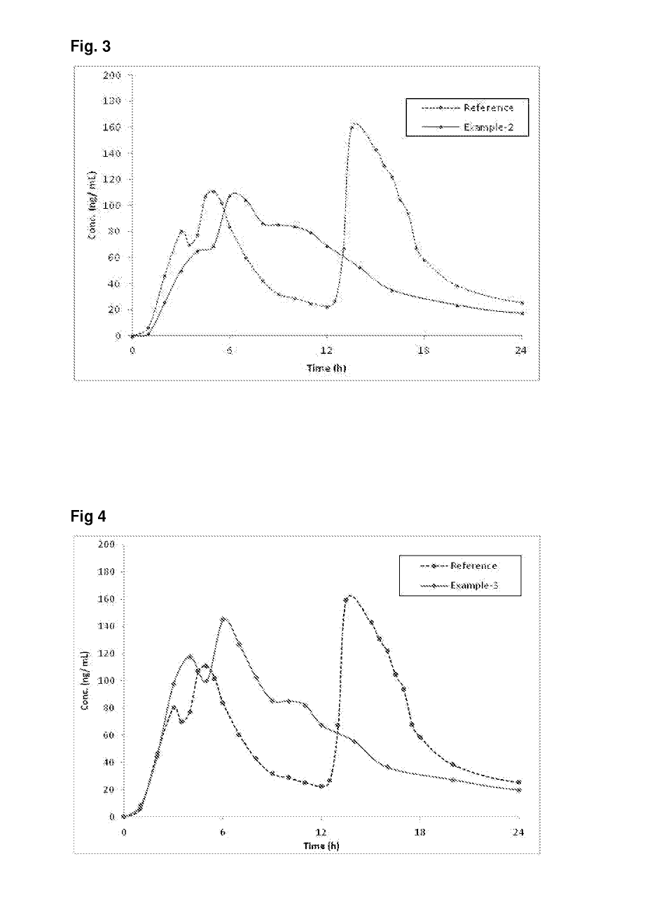

### DETAILED DESCRIPTION OF THE INVENTION

The specification discloses multi component pharmaceutical composition of dronedarone or pharmaceutically acceptable salts thereof which can deliver dronedarone in a controlled manner over an extended period of time. Preferably the multi component composition comprises a prior release component and a controlled release component which maintains a therapeutically effective blood concentration of dronedarone with once a day administration.

The term “composition” as used herein refers to the drug with pharmaceutically acceptable excipients. This includes orally administrable formulations as well as formulations administrable by other means. The term composition can be used interchangeably with the term formulation or dosage form.

As used herein the term dronedarone includes all forms of dronedarone or pharmaceutically acceptable salts, esters, solvates, hydrates, metabolites, prodrugs or isomers thereof. The most preferred form is dronedarone hydrochloride.

Dronedarone may be used in a dose range of from about 50 mg to about 1600 mg.

In one aspect the invention may use dronedarone or pharmaceutically acceptable salts thereof up to 800 mg.

One embodiment discloses a multi component composition of dronedarone comprising a prior release component and a controlled release component wherein prior release component comprises about 20% to about 60% by weight of dronedarone based on total amount of dronedarone in the multi component composition.

Multi component composition of dronedarone may comprise of one or more prior release component comprising dronedarone, one or more controlled release component comprising dronedarone or one or more controlled release component which is free from dronedarone.

Preferably the multi component composition of dronedarone may comprise of one or more prior release component and one or more controlled release component.

Also preferably multi component composition of dronedarone may comprise of one or more controlled release component comprising dronedarone and one or more controlled release polymer, one or more controlled release component which is free from dronedarone and optionally a prior release component.

Further the multi component composition may also comprise of another component which is free from dronedarone optionally comprising control release polymer.

“Controlled release” used herein is defined to mean component that release the drug at a controlled rate and provide plasma concentrations of the drug that remain controlled with time within the therapeutic range of the drug over an extended period of time preferably up to 20 hours or more. When used in association with the dissolution profiles discussed herein, the term “controlled release” refers to that portion of a dosage form made according to the present invention which delivers active agent over a period of time greater than 1 hour.

“Prior-release” used herein is defined to mean a component which is released before the “controlled release component” in the multicomponent composition. The prior-release component comprises dronedarone and if any, an isomer thereof or a pharmaceutically acceptable salt thereof as a pharmacologically active ingredient, and may further contain pharmaceutically acceptable excipients, if necessary.

The prior release component comprises of about 20% to about 60% by weight of dronedarone based on total amount of dronedarone in the said multi component composition. Preferably, prior release component comprises of about 25% to about 55% of total amount of dronedarone and more preferably about 30% to about 50% of total amount of dronedarone.

The term “component” refers to a physically discrete unit or compartment. The components are in physical contact and form a unitary device or composition. The degree of association is only that which is needed to facilitate the oral consumption of the composition as a single dosage form.

The multi component composition may be in the form of tablets (single layered tablets, multilayered tablets, mini tablets, bioadhesive tablets, floating formulation, caplets, matrix tablets, tablet within a tablet, mucoadhesive tablets, modified release tablets, pulsatile release tablets, gastro retentive tablets and timed release tablets), pellets, beads, granules, spheroids, particles, compact, powders, capsules, microcapsules, tablets in capsules, microspheres, matrix formulations, and microencapsulation.

The term controlled release formulation may be used interchangeably with prolonged release, programmed release, timed release, modified release, site specific release, sustained release, extended release, slow release, pulsatile release, delayed release. The controlled release component can be orally disintegrating extended release formulation, osmotic dosage form, bioadhesive formulation, gastro retentive formulation and other such dosage forms.

The prior release component and the controlled release component may be arranged in any manner as apparent to a person skilled in the art.

The controlled release component of dronedarone comprises of dronedarone and one or more than one controlled release polymer. In accordance with the present invention, the term “polymer” includes single or multiple polymeric substances, which can swell, gel, degrade or erode on contact with an aqueous environment (e.g., water).

The skilled artisan will appreciate that the matrix material can be chosen from a wide variety of materials. The controlled-release polymer may be selected from hydrophilic or hydrophobic polymers, hydrophobic material and combinations thereof.

The controlled release component may further comprise of dronedarone, optionally comprising one or more control release polymers and one or more than one control release coating.

The “hydrophilic polymer” may be selected from but not limited to, selected from the group consisting of a saccharide, a cellulose derivative, gum, a protein, a polyvinyl derivative, a hydrophilic polymethacrylate copolymer, a polyethylene derivative, a carboxyvinyl copolymer and a mixture thereof. The saccharide is at least one selected from dextrin, polydextrin, dextran, pectin and a pectin derivative, alginate, polygalacturonic acid, xylan, arabinoxylan, arabinogalactan, starch, hydroxypropyl starch, amylase and amylopectin; the cellulose derivative is at least one selected from hydroxypropylmethylcellulose, hydroxypropylcellulose, hydroxymethylcellulose, hydroxyethylcellulose, methylcellulose, sodium carboxymethylcellulose and hydroxyethylmethylcellulose; the gum is at least one selected from guar gum, locust bean gum, tragacanth, carrageenan, gum acacia, gum arabic, gellan gum and xanthan gum; the protein is at least one selected from gelatin, casein and zein; the polyvinyl derivative is at least one selected from polyvinyl alcohol, polyvinyl pyrrolidone and polyvinylacetaldiethylaminoacetate; the hydrophilic polymethacrylate copolymer is at least one selected from a poly(butyl methacrylate, (2-dimethylaminoethyl)methacrylate, methyl methacrylate) copolymer, a poly(methacrylate, methyl methacrylate) copolymer and a poly(methacrylate, ethyl acrylate) copolymer; the polyethylene derivative is at least one selected from polyethylene glycol and polyethylene oxide; and the carboxyvinyl polymer is carbomer.

The “hydrophobic polymers” may be selected from, but not limited to, polyvinyl acetate dispersion, ethyl cellulose, cellulose acetate, cellulose propionate (lower, medium or higher molecular weight), cellulose acetate propionate, cellulose acetate butyrate, cellulose acetate phthalate, cellulose triacetate, acrylic acid and methacrylic acid copolymers, methyl methacrylate, methyl methacrylate copolymers, ethoxyethyl methacrylate, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid anhydride), polymethacrylate, polyacrylamide, poly(methacrylic acid anhydride), and glycidyl methacrylate copolymers or mixtures thereof.

The “hydrophobic material” may be selected from the group consisting of consisting of fatty acid and fatty acid ester, fatty acid alcohol, wax, inorganic material, and a mixture thereof. The fatty acid or fatty acid ester is at least one selected from glycerylpalmitostearate, glyceryl stearate, glycerylbehenate, cetylpalmitate, glycerylmonooleate and stearic acid; the fatty acid alcohol is at least one selected from cetostearyl alcohol, cetyl alcohol and stearyl alcohol; the wax is at least one selected from carnauba wax, beeswax and microcrystalline wax; and the inorganic material is at least one selected from talc, precipitated calcium carbonate, calcium hydrogen phosphate, zinc oxide, titanium oxide, kaolin, bentonite, montmorillonite and veegum.

The amount of control release polymers that may be used in the composition of the invention is in the range of about 2% to about 90% by weight of the composition. Preferably from about 10% to about 70% by weight of the composition and more preferably from about 15% to about 50% by weight of the composition.

The term “pharmaceutically-acceptable excipients” as used herein includes any physiologically inert, pharmacologically inactive material known to one skilled in the art, which is compatible with the physical and chemical characteristics of dronedarone.

Another embodiment discloses a multi component composition of dronedarone comprising: a) at least one controlled release component comprising dronedarone and control release polymer; b) at least one component comprising control release polymer, characterized in that the component is free of dronedarone; an optional prior release component coated on components a) and b) comprising dronedarone.

Another embodiment discloses a multi component composition of dronedarone comprising: a) prior release component comprising dronedarone and pharmaceutically acceptable excipient wherein prior release component comprises about 20% to about 60% by weight of dronedarone based on total amount of dronedarone in the multi component composition; b) controlled release component comprising control release polymer; c) an optional component comprising controlled release polymer between the components a) and b)

The multi component composition may further contain one or more pharmaceutically acceptable excipients such as binders; diluents; lubricants; disintegrating agents; glidants; stabilizers; osmotic agents; dissolution enhancing agents; pH modifiers, and surface active agents.

Examples of binders include, potato starch; pregelatinized starch; modified starch; gelatin; wheat starch; corn starch; celluloses such as methyl cellulose, hydroxypropyl cellulose, hydroxyethyl cellulose, hydroxypropylmethyl cellulose, ethyl cellulose and sodium carboxy methyl cellulose; hydroxypropyl Starch, polymethacrylates; carbomers; natural gums such as acacia, alginic acid and guar gum; lactose (anhydrous, monohydrate, spray dried); liquid glucose; dextrin; sodium alginate; kaolin; povidone; syrup; polyethylene oxide; polyvinyl pyrrolidone; poly vinyl alcohol; poly-N-vinyl amide; polyethylene glycol; sucrose; polydextrose; gelatin; poly propylene glycol; tragacanth; ceratonia; glycerylbehenate; hydrogenated vegetable oil; zein; castor oil; paraffin; higher aliphatic alcohols; higher aliphatic acids; long chain fatty acids; fatty acid esters; agar; chitosan; maltodextrin; magnesium aluminum silicate; inulin and wax-like materials such as fatty alcohols, fatty acid esters, fatty acid glycerides, hydrogenated fats, hydrocarbons, stearic acid; Copovidone; dextrates, sunflower oil and stearyl alcohol.

Examples of diluents include microcrystalline cellulose; lactose, cellulose powdered, cellulose silicified, cellulose acetate, methyl cellulose, microcrystalline lactose; dibasic or tribasic calcium phosphate; saccharides; confectioner's sugar; compressible sugar; confectioner's sugar; sugar spheres; dextrates; dextrin; dextrose; fructose; maltose; sodium chloride; lactitol; maltodextrin; mannitol; sucrose; fructose; glycerylpalmitostearate; semithicone; Magnesium aluminum silicate; starch; pregelatinized starch; maltitol; xylitol; erythritol; isomalt; sorbitol; sulfobutylether b-cyclodextrin, polymethacrylates; talc; trehalose; ammonium alginate; calcium carbonate; ethyl cellulose; magnesium carbonate; magnesium oxide and calcium sulphate.

The disintegrating agents include povidone, low-substituted hydroxypropyl cellulose; cross-linked polyvinyl pyrrolidone; cross-linked sodium carboxymethylcellulose; hydroxypropyl starch; sodium starch glycolate; sodium starch glycolate; sodium carboxymethylcellulose; carboxymethyl cellulose calcium; sodium carboxymethyl starch; ion-exchange resins such as polacrillin potassium; microcrystalline cellulose; starches and pregelatinized starch; formalin-casein; clays such as bentonite or veegum; guar gum; celluloses or cellulose derivatives; sodium alginate; calcium alginate; alginic acid; chitosan; magnesium aluminum silicate; colloidal silicon dioxide.

The lubricants may be selected from Mg, Al, Ca or Zn stearate; polyethylene glycol; polyvinyl alcohol; glycerylbehenate; glycerylmonostearate; Glycerylpalmitostearate; potassium benzoate; sodium benzoate; mineral oil; sodium stearylfumarate; palmitic acid, myristic acid; stearic acid; hydrogenated vegetable oil; hydrogenated castor oil; talc; hydrogenated soybean oil; stearyl alcohol; leucine; sodium lauryl sulfate; ethylene oxide polymers; poloxamer; octyldodecanol; Sodium stearylfumarate and colloidal silica.

The stabilizers may be selected from naturally occurring as well as synthetic phospholipids, their hydrogenated derivatives and mixtures thereof; organic acids like acetic acid, tartaric acid, citric acid, fumaric acid, lactic acid, and mixtures thereof sphingolipids and glycosphingolipids; physiological bile salts such as sodium cholate, sodium dehydrocholate, sodium deoxycholate, sodium glycocholate and sodium taurocholate; saturated and unsaturated fatty acids or fatty alcohols; ethoxylated fatty acids or fatty alcohols and their esters and ethers; alkylaryl-polyether alcohols such as tyloxapol; esters and ethers of sugars or sugar alcohols with fatty acids or fatty alcohols; acetylated or ethoxylated mono- and diglycerides; synthetic biodegradable polymers like block co-polymers of polyoxyethylene and polyoxypropyleneoxide; ethoxylatedsorbitanesters or sorbitanethers; amino acids, polypeptides and proteins such as gelatine and albumin; or combination thereof.

The glidants may be selected from magnesium trisilicate; powdered cellulose; starch; talc; tribasic calcium phosphate; calcium silicate; magnesium silicate; magnesium trisilicate; colloidal silicon dioxide; and silicon hydrogels.

Dissolution enhancing agents may be selected from, but are not limited to, organic acids, inorganic acids or combination thereof. The organic acids include, but not limited to citric acid, fumaric acid, malic acid, maleic acid, tartaric acid, succinic acid, oxalic acid, aspartic acid, mandelic acid, glutaric acid, and glutamic acid. The inorganic acids include but not limited to hydrochloric acid, phosphoric acid, nitric acid, and sulfuric acid.

Other agents like pH modifiers such as acetic acid/alkali metal acetate, fumaric acid/alkali metal fumarate, succinic acid/alkali metal succinate, citric acid/alkali metal citrate, tartaric acid/alkali metal tartrate, lactic acid/alkali metal lactate, maleic acid/alkali metal maleate, methanesulphonic acid/alkali metal methanesulphonate, monoalkali metal phosphate, the alkali metal in each of the above salts being, for example, sodium or potassium, etc may also be added in the multicomponent composition.

The surface active agents used may be hydrophilic, hydrophobic or combination thereof. Hydrophilic surfactants may be either ionic or non-ionic.

Suitable hydrophilic ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; ammonium lauryl sulfate, sodium lauryl sulfate, sodium myreth sulfate, dioctyl sodium sulfosuccinate, perfluorooctanesulfonate, perfluorobutanesulfonate, alkyl benzene sulfonates, alkyl aryl ether phosphate, alkyl ether phosphate, alkyl carboxylates like, fatty acid salts, sodium stearate, sodium lauroylsarcosinate, octenidinedihydrochloride, cetyltrimethylammonium bromide (CTAB) or hexadecyltrimethyl ammonium bromide, cetyltrimethylammonium chloride (CTAC), cetylpyridinium chloride (CPC), polyethoxylated tallow amine (POEA), benzalkonium chloride (BAC), benzethonium chloride (BZT), 5-Bromo-5-nitro-1,3-dioxane, dimethyldioctadecylammonium chloride, dioctadecyldimethylammonium bromide (DODAB), cocamidopropylbetaine, cocamidopropylhydroxysultaine and mixtures thereof.

Suitable hydrophilic non-ionic surfactants include alkylglucosides; alkylmaltosides; alkylthioglucosides; lauryl macrogolglycerides; polyoxyalkylene alkyl ethers such as polyethylene glycol alkyl ethers; polyoxyalkylenealkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylenesorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof.

Suitable lipophilic surfactants include, but are not limited to fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

The osmotic agents may be selected from sodium chloride; potassium chloride; magnesium sulfate; magnesium chloride; sodium sulfate; lithium sulfate; urea; inositol; sucrose; lactose (anhydrous, monohydrate, spray dried); glucose; sorbitol; fructose; mannitol; dextrose; magnesium succinate; and potassium acid phosphate, sulfobutylether b-cyclodextrin. The osmotic agents may also be added in the controlled release coating.

The controlled release component may also have a coating which controls the release of dronedarone. The porosity of the coating from controlled release component may be modified by using pore forming agents. The pore forming agents may be polymeric or non-polymeric in nature. Any water soluble material present in the coating which dissolves and forms pores in the coating layer may act as pore forming agents. Pore forming agents may be selected form of potassium salts such as potassium chloride, sodium salts as sodium chloride, calcium salts, magnesium salts, amino acids, weak acids, carbohydrates such as sucrose; mannitol; sorbitol, lactose (anhydrous, monohydrate, spray dried), polymers with amino and/or acid functions or polyvinyl pyrrolidine. Examples are aspargine, glutamine, leucin, neroleucine, meglumine, isoleucine, magnesium citrate, magnesium phosphate, magnesium carbonate, magnesium hydroxide, magnesium oxide.

The plasticizers used in coating include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctylazelate, epoxidisedtallate, triisoctyltrimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutylsebacate.

The lubricants used in coating include Mg, Al or Ca or Zn stearate; polyethylene glycol; polyvinyl alcohol; glycerylbehenate; glycerylmonostearate; Glycerylpalmitostearate; potassium benzoate; sodium benzoate; mineral oil; sodium stearylfumarate; palmitic acid, myristic acid; stearic acid; hydrogenated vegetable oil; hydrogenated castor oil; talc; hydrogenated soybean oil; stearyl alcohol; leucine; sodium lauryl sulfate; ethylene oxide polymers; poloxamer; Octyldodecanol; Sodium stearylfumarate and colloidal silica.

As used herein, the term “bioequivalence” denotes a scientific basis on which two or more pharmaceutical compositions containing same active ingredient are compared with one another. “Bioequivalence” means the absence of a significant difference in the rate and extent to which the active agent in pharmaceutical equivalents or pharmaceutical alternatives becomes available at the site of action when administered in an appropriately designed study. Bioequivalence can be determined by an in vivo study comparing a pharmacokinetic parameter for the two compositions. Parameters often used in bioequivalence studies are Tₘₐₓ (time at which the highest drug concentration (Cₘₐₓ) occurs), Cₘₐₓ (highest plasma drug concentration observed), AUC₀₋ₜ. (total plasma exposure of a drug over a given time period) In the present context, substantial bioequivalence of two compositions is established by 90% confidence intervals (CI) of between 0.80 and 1.25 for AUC₍₀₋₂₄₎ and Cₘₐₓ.

Yet another embodiment discloses a multi component composition of dronedarone comprising: a) prior release component comprising dronedarone and pharmaceutically acceptable excipient wherein prior release component comprises about 20% to about 60% by weight of dronedarone based on total amount of dronedarone in the multi component composition; b) controlled release component comprising control release polymer; c) an optional component comprising controlled release polymer between the components a) and b) and demonstrates a plasma exposure of dronedarone over a given time period which is equivalent to two immediate release tablet of dronedarone.

Yet another embodiment discloses a multi component composition of dronedarone comprising a prior release component and a controlled release component wherein prior release component comprises about 20% to about 60% by weight of dronedarone based on total amount of dronedarone in the multi component composition and demonstrates a plasma exposure of a dronedarone over a given time period which is equivalent to two immediate release tablet of dronedarone

Yet another embodiment discloses a multi component composition of dronedarone comprising: a) at least one controlled release component comprising dronedarone and control release polymer; b) at least one component comprising control release polymer, characterized in that the component is free of dronedarone; c) an optional prior release component coated on components a) and b) comprising dronedarone and demonstrates a plasma exposure of dronedarone over a given time period which is equivalent to two immediate release tablet of dronedarone.

The multi component composition may be manufactured by various methods known in the art such as by dry granulation, slugging, roller compaction, wet granulation (using aqueous/non aqueous solvents), melt granulation, solid dispersion, direct compression, double compression, extrusion spheronization, layering, High shear mixture granulation, Fluid bed granulation, spray drying, steam granulation, moisture activated dry granulation, moist granulation, thermal adhesion granulation, foam granulation and the like. Compaction of the blend into coprimate may be carried out using a slugging technique or roller compaction. The milling of the granules may be carried out according to conventional milling methods.

The solvent which may be used for manufacturing the composition may be aqueous, non-aqueous or combination thereof.

The coating operation may be conducted in standard equipment such as a fluid bed coater, a wurster coater or a rotary bed coater. The controlled release coating may be aqueous, non-aqueous or combination of the two.

The invention is not to be limited in scope by the specific embodiments described herein. Indeed, various modifications of the invention in addition to those described herein will become apparent to those skilled in the art from the foregoing description. Such modifications are intended to fall within the scope of the appended claims.

### Example 1

| S. No | Ingredient | Qty (mg/tab) |
|---|---|---|
| Controlled Release Component | | |
| Intragranular | | |
| 1 | Dronedarone HCL eqtoDronedarone 400 mg | 428.085 |
| 2 | Co-Povidone | 125.000 |
| 3 | Lactose M (Pharmatose 200 M) | 7.500 |
| 4 | Monobasic Potassium Phosphate | 10.000 |
| 5 | Maize Starch | 10.000 |
| 6 | HMPC E4M CR | 35.000 |
| 7 | Crospovidone | 5.000 |
| 8 | HPMC K100 LVCR | 57.5 |
| 9 | Dichloromethane | q.s. |
| Extragranular | | |
| 10 | Lactose Monohydrate | 11.15 |
| 11 | HMPC E4M CR | 30.0 |
| 12 | HPMC K100 LVCR | 55.0 |
| 13 | Crospovidone | 2.50 |
| 14 | Colloidal Silicon Dioxide | 5.00 |
| 15 | Magnesium Stearate | 2.50 |
| Total (A) | | 785.00 |
| Prior Release Component | | |
| Intragranular | | |
| 16 | DronedaroneHClEqtoDronedarone 400 mg | 427.50 |
| 17 | Co-povidone | 150.00 |
| 18 | Maize Starch | 34.00 |
| 19 | Crospovidone | 10.00 |
| 20 | Lactose Monohydrate (Pharmatose 200 M) | 28.50 |
| 21 | Dichloromethane | q.s. |
| Extragranular | | |
| 22 | Crospovidone | 16.00 |
| 23 | Lactose Monohydrate (DCL-11) | 24.00 |
| 24 | Colloidal Silicon Dioxide | 5.00 |
| 25 | Magnesium Stearate | 5.00 |

| | |
|---|---|
| Total (B) | 700.00 |
| Total (A) + (B) | 1485.00 |

### Brief Manufacturing Procedure:

### Controlled Release Component

1. The weighed quantity of Dronedarone HCl along with Co-Povidone, Maize Starch, Crospovidone, Lactose Monohydrate, HMPC E4M CR and HMPC K100 LVCR is sifted through 30# SS Sieve.
2. Monobasic Potassium Phosphate is sifted through 100 # SS Sieve.
3. The blend of step 1 and step 2 is loaded in rapid mixer granulator and dry mixed it for 5 minutes.
4. The blend of step 3 is granulated using sufficient quantity of Dichloromethane with suitable granulation parameters.
5. The wet granules of step 4 are dried in tray dryer at 60° C. inlet temperature till its LOD reaches
6. The dried granules of step 5 are passed through 20 # SS Sieve.
7. The extragranular quantity of Lactose Monohydrate, Crospovidone, and Colloidal silicon dioxide is passed through 40 #SS Sieve and mixed well with granules of step 6.
8. The blend of step 7 is lubricated with Magnesium Stearate (passed through 40# SS Sieve) for mix for 3 minutes.

### Prior Release Component

9. The weighed quantity of Dronedarone HCl, Co-povidone, Maize starch, Crospovidone and Lactose Monohydrate (Pharmatose 200M) is sifted through 30# SS Sieve.
10. The blend of step 9 is loaded in rapid mixer granulator and dry mixed for 5 minutes.
11. The blend of step 10 is granulated using sufficient quantity of Dichloromethane with suitable granulation parameters.
12. The wet granules of step 11 are passed through 12 # SS Sieve and dried in tray dryer at 60° C. inlet temperature till its LOD reaches between 1.5-2.5% w/w at 105° C. up to constant weight.
13. The dried granules of step 12 are passed through 20 # SS Sieve.
14. The extragranular quantity of Crospovidone, Lactose Monohydrate (DCL-11) and Colloidal Silicon Dioxide is passed through 40 #SS Sieve and mixed well with granules of step 13.
15. The blend of step 14 is lubricated with Magnesium Stearate (passed through 40# SS Sieve) for mix for 3 minutes.
16. The blend of step 8 and step 15 is compressed in to bilayer tablet using 17.00×12.00 mm oval shape punch using suitable physical parameters.

### Example 2

| S. No | Ingredient | Qty (mg/tab) |
|---|---|---|
| Controlled Release Component | | |
| Intragranular | | |
| 1 | Dronedarone HCL eqtoDronedarone 400 mg | 430.00 |
| 2 | Co-Povidone | 125.00 |
| 3 | Monobasic Potassium Phosphate | 25.00 |
| 4 | Crospovidone | 25.00 |
| 5 | HMPC K100 LVCR | 50.00 |
| 6 | Dichloromethane | q.s. |
| Extragranular | | |
| 7 | Polyethylene Oxide | 60.00 |
| 8 | HPMC K100M CR | 15.00 |
| 9 | Crospovidone | 5.00 |
| 10 | Colloidal silicon dioxide | 10.00 |
| 11 | Magnesium Stearate | 5.00 |
| Total core | | 750.00 |
| Functional Coat | | |
| 12 | Ethyl Cellulose | 7.20 |
| 13 | HPMC 5 cps | 16.80 |
| 14 | Triethylcitrate | 6.00 |
| 15 | IPA | q.s |
| 16 | Dichloromethane | q.s. |
| Total (A) | | 780.00 |
| Prior Release Component | | |
| Intragranular | | |
| 17 | DronedaroneHClEqtoDronedarone 400 mg | 427.0 |
| 18 | Co-povidone | 150.0 |
| 19 | Maize Starch | 34.0 |
| 20 | Crospovidone | 10.0 |
| 21 | Lactose Monohydrate (Pharmatose 200 M) | 28.5 |
| 22 | Dichloromethane | q.s. |
| Extragranular | | |
| 23 | Crospovidone | 16.0 |
| 24 | Lactose Monohydrate (DCL 11) | 24.0 |
| 25 | Colloidal Silicon Dioxide | 5.0 |
| 26 | Magnesium Stearate | 5.0 |

| | |
|---|---|
| Total (B) | 700.0 |
| Total (A) + (B) | 1480.0 |

### Brief Manufacturing Procedure:

### Controlled Release Component

1. The weighed quantity of Dronedarone HCL, Co-Povidone, Crospovidone, and HMPC K100 LVCR is sifted through 30# SS Sieve.
2. Monobasic Potassium Phosphate is sifted through 100 # SS Sieve.
3. The blend of step 1 and step 2 is loaded in rapid mixer granulator and dry mixed for 5 minutes.
4. The blend of step 3 is granulated using sufficient quantity of Dichloromethane with suitable granulation parameters.
5. The wet granules of step 4 are dried in tray dryer at 60° C. inlet temperature till its LOD reaches.
6. The dried granules of step 5 are passed through 20 # SS Sieve.
7. The extragranular quantity of polyethylene oxide, HPMC K100M CR, Crospovidone and Colloidal Silicon Dioxide is passed through 40 #SS Sieve and mixed well with granules of step 6.
8. The blend of step 7 is lubricated with Magnesium Stearate (passed through 40# SS Sieve) for mix for 3 minutes.
9. The lubricated blend of step 8 is compressed in to tablet using 11.50 mm round shape punch using suitable physical parameters.
10. The weighed quantity of Ethyl cellulose, HPMC and Triethylcitrate is dissolved in IPA and Dichloromethane mixture (50:50) and stirred for 45 minutes.
11. The compressed tablets of step 9 are coated with dispersion prepared in step 10.

### Prior Release Component

12. The weighed quantity of Dronedarone HCl, Co-povidone, Maize starch, Crospovidone and Lactose Monohydrate (Pharmatose 200M) is sifted through 30# SS Sieve.
13. The blend of step 12 is loaded in rapid mixer granulator and dry mixed for 5 minutes.
14. The blend of step 13 is granulated using sufficient quantity of Dichloromethane with suitable granulation parameters.
15. The wet granules of step 14 are passed through 12 # SS Sieve and dried in tray dryer at 60° C. inlet temperature till its LOD reaches between 1.5-2.5% w/w at 60° C. up to constant weight.
16. The dried granules of step 15 are passed through 20 # SS Sieve.
17. The extragranular quantity of Crospovidone, Lactose Monohydrate (DCL-11) and Colloidal Silicon Dioxide is passed through 40 #SS Sieve and mixed well with granules of step 16.
18. The blend of step 17 is lubricated with Magnesium Stearate (passed through 40# SS Sieve) for mix for 3 minutes.
19. The blend of step 11 and step 17 is compressed in to bilayer tablet using 17.00×12.00 mm oval shape punch using suitable physical parameters.

### Example 3

| S. No | Ingredient | Qty (mg/tab) |
|---|---|---|
| Controlled Release Component | | |
| Intragranular | | |
| 1 | Dronedarone HCL eqtoDronedarone 400 mg | 430.0 |
| 2 | Co-Povidone | 125.0 |
| 3 | Monobasic Potassium Phosphate | 25.0 |
| 4 | Crospovidone (XL-10) | 25.0 |
| 5 | HMPC K100 LVCR | 50.0 |
| 6 | Dichloromethane | q.s. |
| Extragranular | | |
| 7 | Polyethylene Oxide | 60.0 |
| 8 | HPMC K 100M CR | 15.0 |
| 9 | Crospovidone | 5.0 |
| 10 | Colloidal silicon dioxide | 10.0 |
| 11 | Magnesium Stearate | 5.0 |
| Total (A) | | 750.0 |
| Dronedarone Free Component | | |
| 12 | HPMC K100 MCR | 38.48 |
| 13 | Polyox WSR 303 | 38.48 |
| 14 | Lactose Monohydrate (DCL-11) | 20.00 |
| 15 | Colloidal silicon dioxide | 1.92 |
| 16 | Magnesium Stearate | 1.16 |
| Total (A1) | | 100.00 |
| Prior Release Component | | |
| Intragranular | | |
| 17 | Dronedarone HCL eqtoDronedarone 400 mg | 427.5 |
| 18 | Co-Povidone | 150.0 |
| 19 | Maize starch | 34.0 |
| 20 | Crospovidone | 10.0 |
| 21 | Lactose monohydrate (Pharmatose 200 M) | 28.5 |
| 22 | Dichloromethane | q.s. |
| Extragranular | | |
| 23 | Crospovidone | 16.0 |
| 24 | Lactose Monohydrate (DCL-11) | 24.0 |
| 25 | Colloidal silicon dioxide | 5.0 |
| 26 | Magnesium Stearate | 5.0 |

| | |
|---|---|
| Total (B) | 700.0 |
| Total (A + A1) + (B) | 1550.0 |

### Brief Manufacturing Procedure:

### Controlled Release Component

1. The weighed quantity of Dronedarone HCL, Co-Povidone, Crospovidone, and HMPC K100 LVCR is sifted through 30# SS Sieve.
2. Monobasic Potassium Phosphate is sifted through 100 # SS Sieve.
3. The blend of step 1 and step 2 is loaded in rapid mixer granulator and dry mixed for 5 minutes.
4. The blend of step 3 is granulated using sufficient quantity of Dichloromethane with suitable granulation parameters.
5. The wet granules of step 4 are dried in tray dryer at 60° C. inlet temperature till its LOD reaches.
6. The dried granules of step 5 are passed through 20 # SS Sieve.
7. The extragranular quantity of polyethylene oxide, HPMC K100M CR, Crospovidone and Colloidal silicon dioxide is passed through 40 #SS Sieve and mixed well with granules of step 6.
8. The blend of step 7 is lubricated with Magnesium Stearate (passed through 40# SS Sieve) for mix for 3 minutes.

### Dronedarone Free Component

9. The weighed quantity of HPMCK100MCR, Polyox WSR 303, Lactose Monohydrate, and Colloidal silicon dioxide is sifted through 40# SS Sieve.
10. The blend of step 9 is lubricated with Magnesium Stearate (passed through 40# SS Sieve) for mix for 3 minutes.
11. The lubricated blend of step 8 and Step 10 is compressed in to bilayer tablet using 11.50 mm oval shape punch using suitable physical parameters.

### Prior Release Component

12. The weighed quantity of Dronedarone HCl, Co-povidone, Maize starch, Crospovidone and Lactose Monohydrate (Pharmatose 200M) is sifted through 30# SS Sieve.
13. The blend of step 12 is loaded in rapid mixer granulator and dry mixed for 5 minutes.
14. The blend of step 13 is granulated using sufficient quantity of Dichloromethane with suitable granulation parameters.
15. The wet granules of step 14 are passed through 12 # SS Sieve and dried in tray dryer at 60° C. inlet temperature till its LOD reaches between 1.5-2.5% w/w at 60° C. up to constant weight.
16. The dried granules of step 15 are passed through 20 # SS Sieve.
17. The extragranular quantity of Crospovidone, Lactose Monohydrate (DCL-11) and Colloidal Silicon Dioxide is passed through 40 #SS Sieve and mixed well with granules of step 16.
18. The blend of step 17 is lubricated with Magnesium Stearate (passed through 40# SS Sieve) for mix for 3 minutes.
19. The blend of step 11 and step 17 is compressed in to bilayer tablet using 17.00×12.00 mm oval shape punch using suitable physical parameters.

### Dissolution Profile:

The dissolution of the controlled release formulation of example 1, 2, and 3 was carried out in USP basket method of 100 rpm in 1000 ml phosphate buffer at pH 4.5 at 37° C.

TABLE 1
Drug release Profile:

| Time (hr) | Example 1 | Example 2 | Example 3 |
|---|---|---|---|
| 0 | 0 | 0 | 0 |
| 1 | 22 | 38.8 | 23.6 |
| 2 | 41 | 45.3 | 43.4 |
| 4 | 56.2 | 58.1 | 61.4 |
| 6 | 64.7 | 72.5 | 72.9 |
| 8 | 73.7 | 83.1 | 81.1 |
| 10 | 79.1 | 92.4 | 85.4 |
| 12 | 82 | 96.3 | 89.7 |
| 14 | 87.9 | 97.7 | 92.9 |
| 16 | 89.8 | 98.3 | 96.3 |
| 18 | 91.2 | 99.2 | 98.5 |
| 20 | 90.2 | 98.7 | 99.1 |

### Summary of Pharmacokinetic Studies

A comparison of the bioavailability of multi component composition of dronedarone prepared according to example 1, 2, and 3 with Multaq® 400 mg immediate release tablet was carried out in 08 healthy adult male volunteers under standard fed conditions.

Results of pharmacokinetic studies under fed conditions are as indicated in Table 1, 2, and 3 below:

TABLE 2
Results of pharmacokinetic studies of multi component
composition of dronedarone prepared as Example 1
under fed conditions with twice daily (BID) Multaq ®
400 mg immediate release tablet.

| | AUC₍₀₋ₜ₎ | | | |
|---|---|---|---|---|
| Treatment | (ng · h/ml) | T/R (AUC) | Cₘₐₓ | T/R (Cₘₐₓ) |
| Example-1 | 1431.0 | 85.0 | 136.69 | 82.9 |
| Multaq ® | 1684.0 | | 164.88 | |

Example 1 shows a relative bioavailability is 85% and adequately covers the Cₘₐₓ of 1^{st} dose of Multaq T/R for Cₘₐₓ is 82% for the highest IR peak indicating better tolerability of the controlled release composition on long term usage.

TABLE 3
Results of pharmacokinetic studies of multi component
composition of dronedarone prepared as Example 2
under fed conditions with twice daily (BID) Multaq ®
400 mg immediate release tablet.

| | AUC₍₀₋ₜ₎ | | | |
|---|---|---|---|---|
| Treatment | (ng · h/ml) | T/R (AUC) | Cₘₐₓ | T/R (Cₘₐₓ) |
| Example-2 | 1433.3 | 85.1 | 121.76 | 73.8 |
| Multaq ® | 1684.0 | | 164.88 | |

Example 2 shows a relative bioavailability of ˜85%; which adequately covers the Cₘₐₓ of 1^{st} IR dose. T/R for Cₘₐₓ is 73.8% for the highest IR peak indicating better tolerability of the CR formulation on long term usage.

TABLE 4
Results of pharmacokinetic studies of multi component
composition of dronedarone prepared as Example 3
under fed conditions with twice daily (BID) Multaq ®
400 mg immediate release tablet.

| | AUC₍₀₋ₜ₎ | | | |
|---|---|---|---|---|
| Treatment | (ng · h/ml) | T/R (AUC) | Cₘₐₓ | T/R (Cₘₐₓ) |
| Example-3 | 1675.3 | 99.5 | 143.24 | 86.9 |
| Multaq ® | 1684.0 | | 164.88 | |

The relative bioavailability in case of Example-3 is ˜100%. The onset of action or the initial absorption profile (0-4 h) matches to that of first IR dose. T/R for Cₘₐₓ is 86.9% indicating lesser propensity towards peak exposure related side-effects when compared to IR profile in addition to the advantage of less frequent dosing.

1. A multi component composition of dronedarone comprising a prior release component and a controlled release component wherein prior release component comprises about 20% to about 60% by weight of dronedarone based on total amount of dronedarone in the multi component composition.

2. The multi component composition of dronedarone, according to claim 1, wherein prior release component comprises about 30% to about 55% by weight of dronedarone.

3. The multi component composition of dronedarone, according to claim 1, wherein the controlled release component comprises dronedarone and controlled release polymer.

4. The multi component composition of dronedarone, according to claim 3, wherein the component is further coated with a controlled release coating.

5. The multi component composition of dronedarone, according to claim 1, further comprises a component which is free from dronedarone and comprises of controlled release polymer.

6. The multi component composition of dronedarone, according to claim 1 wherein the component is in the form of layers, pellets, granule, spheroids, mini tablets and mixtures thereof

7. The multi component composition of dronedarone, according to claim 1, wherein the composition is selected from a tablet or a capsule.

8. The multi component composition of dronedarone, according to claim 7, wherein the tablet is a bilayer or multilayer tablet or a matrix.

9. The multi component composition of dronedarone comprising:
a) at least one controlled release component comprising dronedarone and control release polymer;
b) at least one component comprising control release polymer, characterized in that the component is free of dronedarone;
c) an optional prior release component coated on components a) and b) comprising dronedarone.

10. The multi component composition of dronedarone, according to claim 9, wherein prior release component comprises about 20% to about 60% by weight of dronedarone based on total amount of dronedarone in the multi component composition

11. The multi component composition of dronedarone comprising:
a) prior release component comprising dronedarone and pharmaceutically acceptable excipient wherein prior release component comprises about 20% to about 60% by weight of dronedarone based on total amount of dronedarone in the multi component composition.
b) controlled release component comprising control release polymer;
c) an optional component comprising controlled release polymer between the components a) and b).

12. The multi component composition of dronedarone, according to claim 11, wherein prior release component comprises about 20% to about 60% by weight of dronedarone based on total amount of dronedarone in the multi component composition.
